(19) United States
(12) Patent Application Publication
(10) Pub. No.: US 2026/0036508 A1
(43) Pub. Date: Feb. 5, 2026

(54) CARTRIDGE FOR STORING TISSUE FOR IMAGING

(71) Applicant: ILLUMISONICS INC., Waterloo (CA)

(72) Inventors: Parsin HAJI REZA, Waterloo (CA); Rocky GANSKE, Waterloo (CA); Michael BISHOP, Waterloo (CA); John MACKEY, Waterloo (CA); Deepak DINAKARAN; Yunqing LI, Waterloo (CA); Jochen SCHWEIZER, Waterloo (CA); Hager GAOUDA, Waterloo (CA)

(73) Assignee: ILLUMISONICS INC., Waterloo, ON (CA)

(21) Appl. No.: 18/993,425

(22) PCT Filed: Jul. 11, 2023
(86) PCT No.: PCT/IB2023/057121

§ 371 Date: Jan. 10, 2025

Related U.S. Application Data
(60) Provisional application No. 63/368,112, filed on Jul. 11, 2022.
(60) Provisional application No. 63/483,556, filed on Feb. 7, 2023.

(51) Int. Cl. G01N 21/03; A61B 10/00; G01N 21/51

(52) U.S. Cl. CPC G01N 21/03; A61B 10/0096; G01N 21/51; G01N 2021/0339

(57) ABSTRACT

A cartridge for storing an un-labelled tissue to be imaged by an optical imaging system may include: a container to store the un-labelled tissue, and to interface with the optical imaging system; an optical substrate, provided on a bottom surface of the container, through which the optical imaging system is configured to image the un-labelled tissue to generate a virtually-stained histological image of the un-labelled tissue; and a lid, provided on a top surface of the container, including a membrane to compress the un-labelled tissue against the optical substrate such that an entire margin of the un-labelled tissue is flat against the optical substrate.

### CROSS-REFERENCE TO RELATED APPLICATIONS

This application claims priority to U.S. Provisional Patent Application No. 63/368,112, filed on Jul. 11, 2022, and to U.S. Provisional Patent Application No. 63/483,556, filed on Feb. 7, 2023, the entirety of each of which is incorporated herein by reference.

### FIELD

The present disclosure relates to a cartridge for storing tissue for imaging, and more particularly to a cartridge for storing an un-labelled tissue to be imaged by an optical imaging system configured to generate a virtually-stained histological image of the un-labelled tissue. However, the embodiments herein are also applicable to molecular diagnostics.

### BACKGROUND

Surgical excision is an integral part of treatment for most solid cancerous tumors, and involves a surgeon resecting the tumor and peritumoral tissue. A “surgical margin” (or “margin”) may refer to the border of the resected tissue. A “negative surgical margin” may refer to a surgical margin that does not overlap with, or that is sufficiently distanced from, a cancerous tumor. A “positive surgical margin” may refer to a surgical margin that overlaps with, or that is not sufficiently distanced from, a cancerous tumor.

During the surgical excision, a surgeon may have difficulty in accurately determining the amount of peritumoral tissue to resect such that the appropriate surgical margin is achieved, and may have difficulty determining whether a negative margin exists. Accordingly, many initial excision surgeries result in a positive margin which results in the need for additional surgery.

After surgical excision of the tissue, histopathologic assessment is performed on the resected tissue to determine whether the margin is negative or positive. Generally, histopathologic assessment of a tissue includes chemically stabilizing the resected tissue with a fixative, embedding the resected tissue in paraffin, slicing the resected tissue, mounting the resected tissue on a glass slide, staining the resected tissue with dyes (e.g., hematoxylin and eosin), and performing light microscopy qualitative analysis of histological images of the stained tissue. The foregoing technique is time-intensive, expensive, error-prone, and cannot be performed intraoperatively.

In some cases, frozen section analysis allows for the intraoperative assessment of surgical margins. Frozen section analysis includes embedding the resected tissue in specialized media, cooling the resected tissue, freezing the resected tissue, slicing the resected tissue, staining the resected tissue, mounting the stained tissue, and analyzing histological images of the stained tissue. The foregoing technique prolongs operating times, and introduces artifacts which deteriorate the cellular morphology and affect the pathological diagnosis of the tissue. In this way, frozen section analysis often includes an unacceptably high false positive rate.

Accordingly, there is a need for techniques for providing intraoperative assessment of surgical margins in an accurate, safe, and time-sensitive manner.

### SUMMARY

Embodiments of the present disclosure relate to, among other things, a cartridge for storing an un-labelled tissue to be imaged by an optical imaging system configured to generate a virtually-stained histological image of the un-labelled tissue. Each of the embodiments disclosed herein may include one or more of the features described in connection with any of the other disclosed embodiments.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIGS. 1A-1M are diagrams of a cartridge for storing an un-labelled tissue.

FIG. 2 is a diagram of a cartridge interfacing with an optical imaging system.

FIG. 3A is a diagram of a cartridge storing an un-labelled tissue including a notch.

FIG. 3B is a diagram of a display including a tissue image and a patient orientation image.

FIG. 4A is a diagram of a paper including orientation marks.

FIG. 4B is a diagram of a cartridge including orientation marks.

FIG. 5A is a diagram of a cartridge including a unique identifier.

FIG. 5B is a diagram of a cartridge including a unique identifier.

FIGS. 6A-6G are diagrams of a pressure assembly.

FIG. 7 is a diagram of a cartridge including fiducials.

FIG. 8 is a diagram of components of an optical imaging system.

FIG. 9 is a flowchart of a process for preparing an un-labelled tissue for imaging.

FIG. 10 is a flowchart of a process for preparing a cartridge for imaging.

FIG. 11 is a flowchart of a process for detecting an interlock between a cartridge and an optical imaging system.

FIG. 12 is a flowchart of a process for imaging a tissue.

FIG. 13 is a flowchart of a process for adjusting an imaging head of an optical imaging system.

FIG. 14 is a flowchart of a process for storing a tissue in a cartridge.

### DETAILED DESCRIPTION

As addressed above, various histological imaging techniques are time-intensive and error-prone, and, in many cases, cannot be performed intraoperatively. As such, intraoperative negative margin confirmation might be exceedingly difficult or impossible in conventional imaging techniques.

The present disclosure provides a cartridge for storing an un-labelled tissue to be imaged by an optical imaging system configured to generate a virtually-stained histological image of the un-labelled tissue. However, the embodiments herein are also applicable to imaging labelled tissue (e.g., molecular imaging using labelled antibodies, molecular imaging using labelled antigens, molecular imaging using labelled oligonucleotides, fluorescence imaging, or the like).

The cartridge includes a container to store the un-labelled tissue, and to interface with the optical imaging system. Further, the cartridge includes an optical substrate through which the optical imaging system is configured to image the un-labelled tissue to generate the virtually-stained histological image of the un-labelled tissue. Further still, the cartridge includes a lid including a membrane to compress the un-labelled tissue against the optical substrate such that an entire margin of the un-labelled tissue is flat against the optical substrate.

During surgery, tissue may be resected from a patient, and the resected and un-labelled tissue may be placed in the cartridge. The cartridge then may be interfaced with the optical imaging system. The optical imaging system may intraoperatively generate a virtually-stained histological image of a true margin of the un-labelled tissue while the cartridge maintains a tissue orientation as resected from the patient. In this way, determination of whether a negative margin exists in a resected tissue sample can be made more accurately, quickly, and intraoperatively, which thereby improves the safety and efficacy of surgical excision of cancerous tumors.

In some embodiments, an “un-labelled tissue” refers to a tissue that is not stained with a stain used in histology. For example, un-labelled tissue is not stained with a stain such as hematoxylin, eosin, an acid dye, a basic dye, a periodic acid-Schiff reaction stain, a Masson's stain, an Alcian blue stain, a Van Gieson stain, a Reticulin stain, a Giemsa stain, a toluidine blue stain, a silver and gold stain, a chrome alum stain, a haemotoxylin stain, an Isamin blue stain, an osmium stain, PAS, T-blue, Congo Red, Crystal Violet, or the like. In some embodiments, a “virtually-stained histological image” refers to an image of an un-labelled tissue that emulates staining. In other words, a virtually-stained histological image depicts how the un-labelled tissue would, or might, appear if stained.

FIG. TA is a diagram of a cartridge for storing an un-labelled tissue. As shown in FIG. TA, the cartridge 100 may include a container 110, an optical substrate 120, a lid 130, a membrane 140, and a cap 150. As further shown in FIG. TA, the cartridge 100 may store an un-labelled tissue 160.

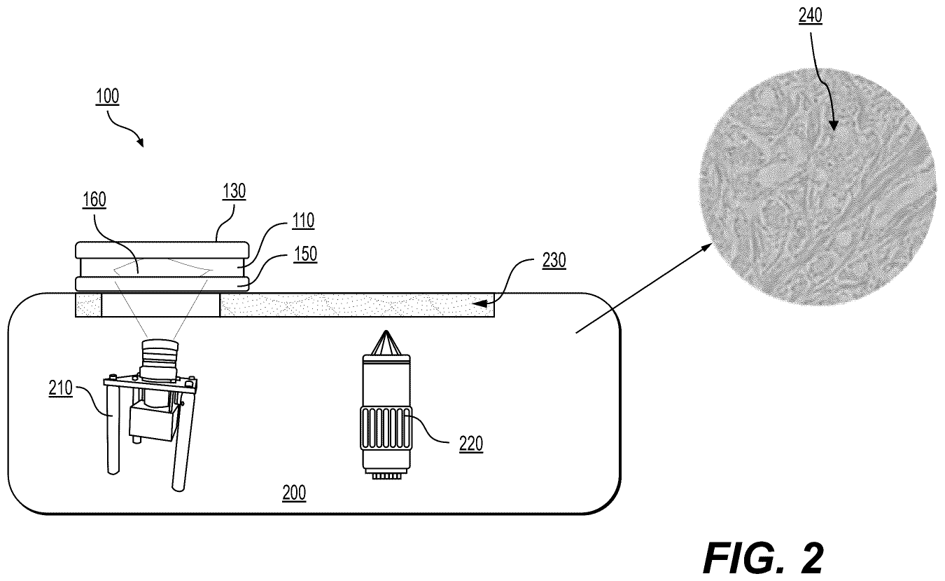

The container 110 may store the un-labelled tissue 160, and may interface with an optical imaging system 200 (shown in FIG. 2) configured to generate a virtually-stained histological image of the un-labelled tissue 160. The container 110 may include a top surface 110-1, a bottom surface 110-2, and a side surface 110-3. The bottom surface 110-2 and the side surface 110-3 of the container 110 may form a cavity in which the un-labelled tissue 160 is placed and stored. The container 110 may be made of any suitable material (e.g., plastic, metal, etc.), and may be any suitable shape (e.g., circular, hexagonal, square, etc.).

The optical substrate 120 may be provided on the bottom surface 110-2 of the container 110, and may permit the optical imaging system 200 to image the un-labelled tissue 160 through the optical substrate 120 to generate a virtually-stained histological image 240, as shown in FIG. 2, of the un-labelled tissue 160. The optical substrate 120 may be formed of any suitable material, and may be any suitable shape. For example, the optical substrate 120 may be comprised of an optically transparent material configured for the wavelengths of operation of the optical imaging system 200. As a particular example, based on the excitation wavelengths of the optical imaging system 200 being in the range of 250 nanometers to 270 nanometers, the optical substrate 120 may be an ultraviolet fused silica or quartz. Moreover, the optical substrate 120 may be configured to allow photons of a particular wavelength to pass based on a particular configuration of the optical imaging system 200.

Additionally, or alternatively, the optical substrate 120 may include an anti-reflective coating. In some implementations, the anti-reflective coating may be single use only, and might not be compatible with sterilization or cleaning protocols. In this way, the single-use nature of the optical substrate 120 might prevent the cartridge 100 from being used multiple times, thereby preventing or reducing a risk of contamination and false negatives or false positives.

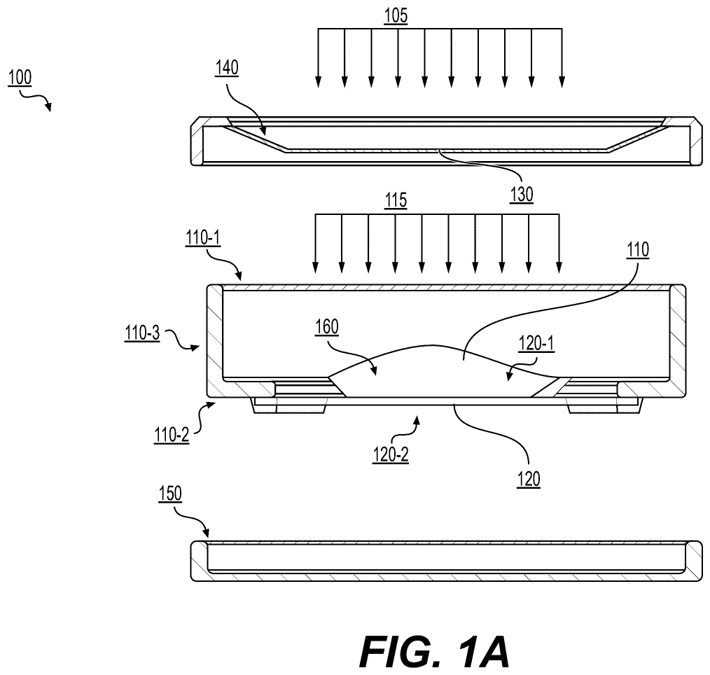

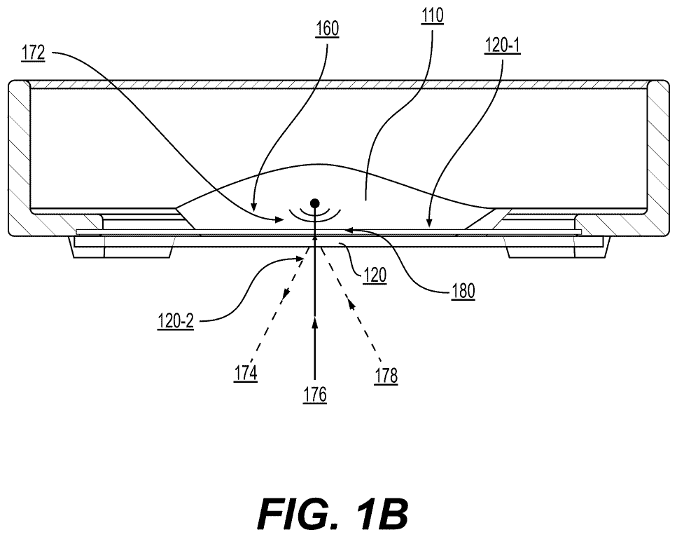

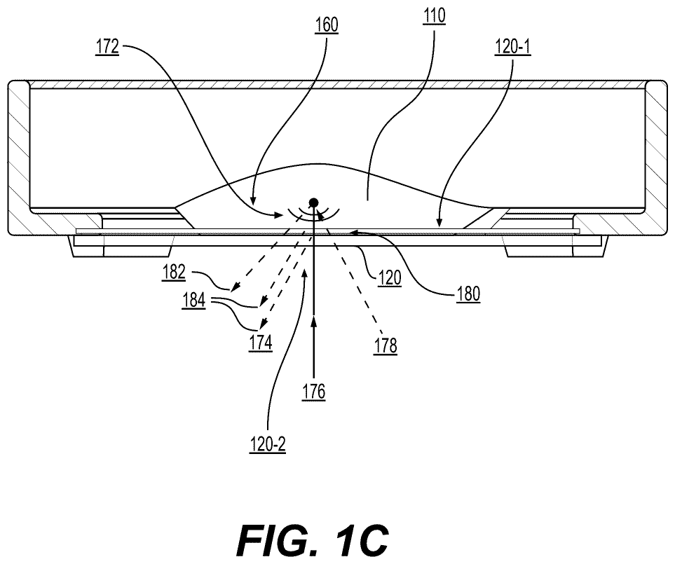

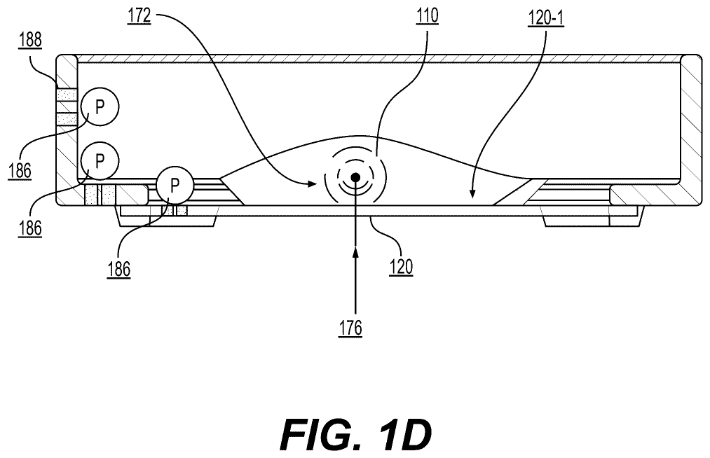

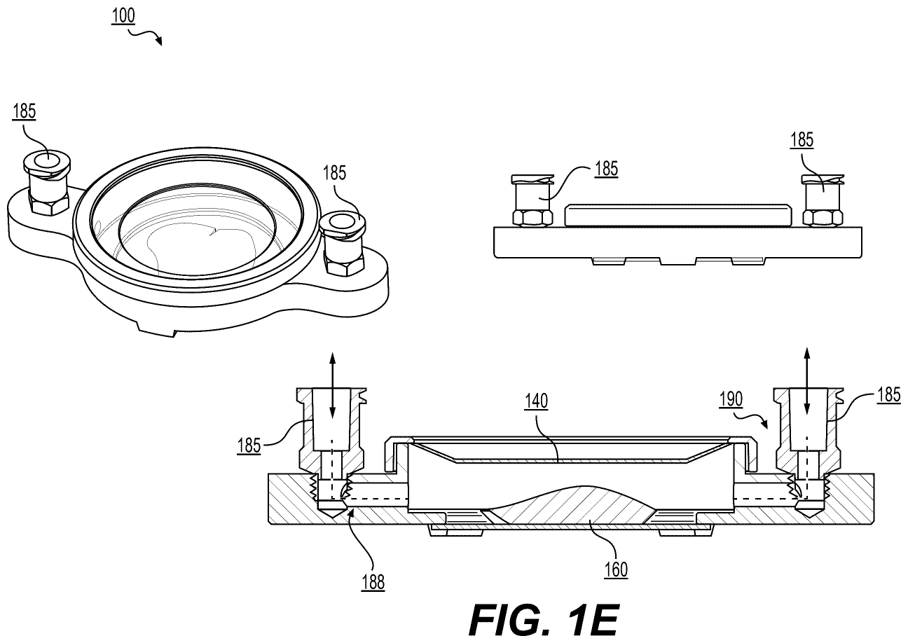

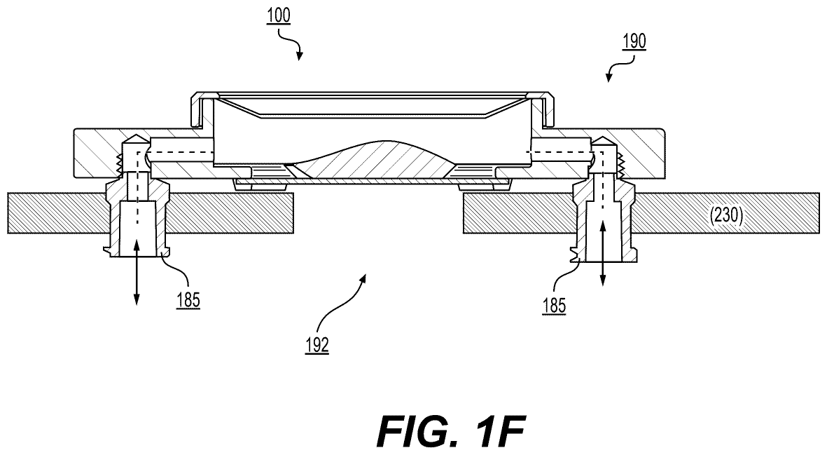

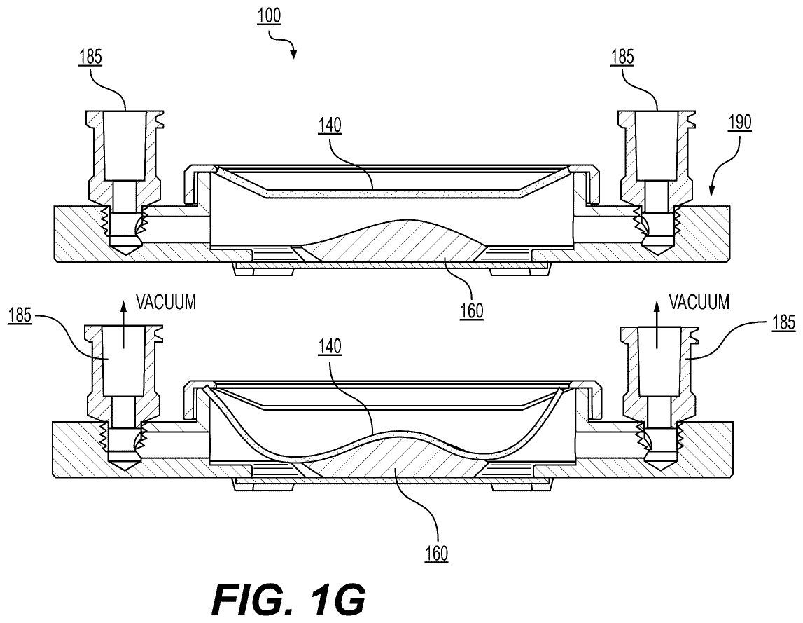

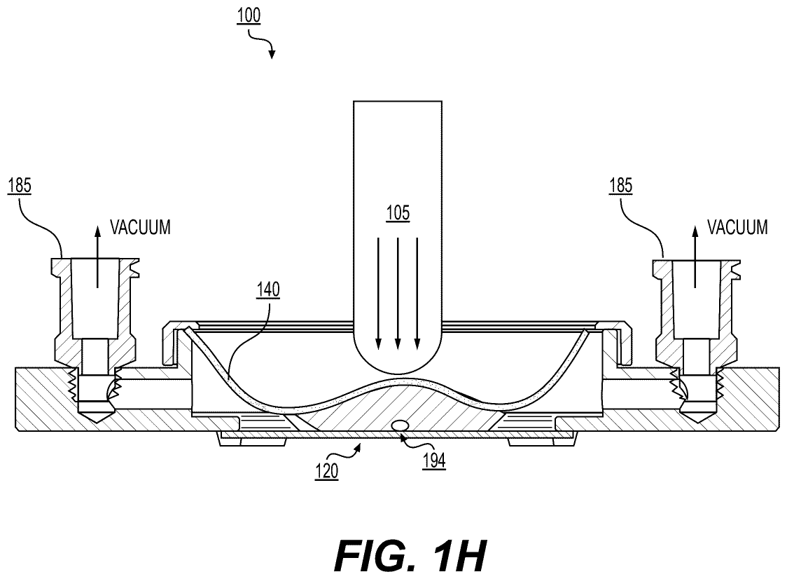

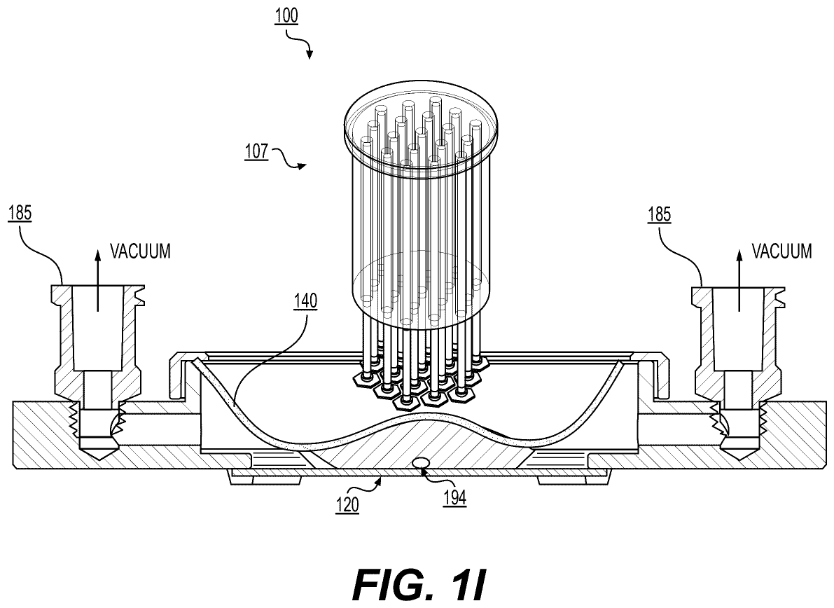

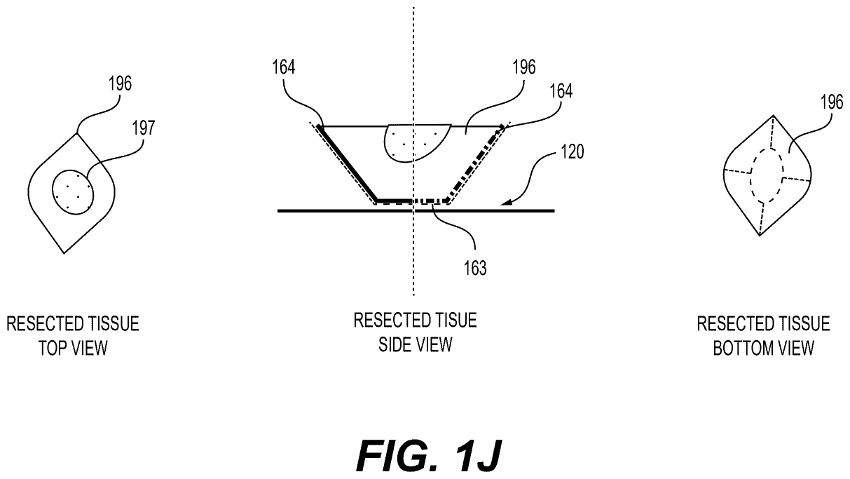

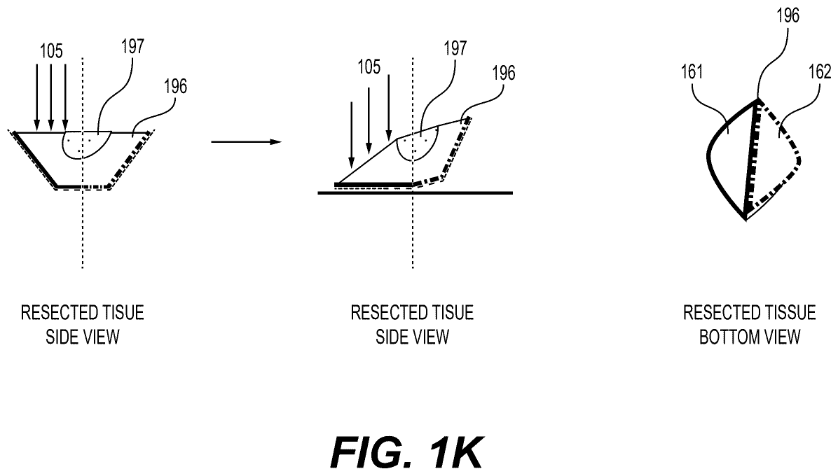

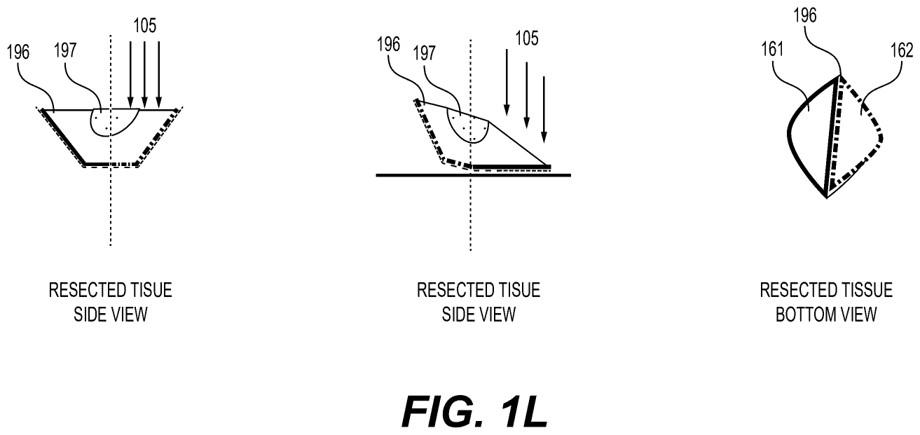

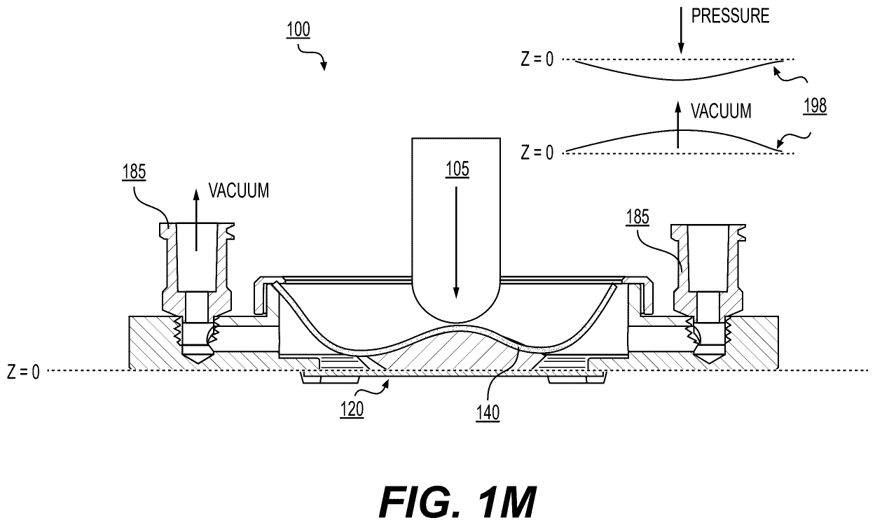

Additionally, or alternatively, the optical substrate 120 may include a Fabry-Pérot etalon 180 (as shown in FIGS. 1B and 1C) to permit the optical imaging system 200 to capture all photo-acoustic data streams. The optical imaging system 200, using the Fabry-Pérot etalon 180, may capture ultrasound propagation data that is used in conjunction with photon absorption remote sensing data streams (e.g., radiative, non-radiative, and scattering) to aid in image reconstruction of an image of the un-labelled tissue 160. The Fabry-Pérot etalon 180 may be provided on a top surface 120-1 of the optical substrate 120 that contacts the un-labelled tissue 160. Alternatively, the Fabry-Pérot etalon 180 may be provided on a bottom surface 120-2 of the optical substrate 120. The Fabry-Pérot Etalon 180 is deposited on surface of the optical substrate. The Fabry-Pérot Etalon 180 can be deposited onto the surface of the glass using thin film deposition techniques. The initial pressure (generated from absorption of excitation light from excitation laser 176) creates an acoustic wave 172 which modulates the thickness of the thin film of the Fabry-Pérot etalon 180, which is detected as a modulation on the detection laser 178. Detection laser collection 174 can be set up to capture the radiative/non-radiative channels 182 at focused depth into tissue as well as the Fabry-Pérot etalon channels 184 at first and second surface of the Fabry-Pérot etalon 180.

Additionally, or alternatively, the cartridge 100 may include a transducer 186 (as shown in FIG. 1D) to permit the optical imaging system 200 to capture an ultrasound data stream. The ultrasound data stream may assist in interrogating signals that are deeper than what might be addressable with photon absorption remote sensing, and may permit the optical imaging system 200 to reconstruct and collect depth scanning information. The transducer 186 may be provided on the optical substrate 120. Alternatively, the transducer 186 may be provided in mechanical contact with the cartridge 100. Additionally, the cartridge 100 may include a liquid buffer. In this case, the ultrasound waves 172 may pass through the liquid buffer to reach the transducer 186.

Additionally, or alternatively, the cartridge 100 may include electrical vias 188 (as shown in FIG. 1D) that permit electrical current to pass through the transducer 186 of the cartridge 100 without casing leaks in the cartridge 100. Further, the electrical vias 188 may interconnect the cartridge 100 to the cartridge plate 230 of the optical imaging system 200. In this case, an electrical loop may provide an interlock that prevents operation of the optical imaging system 200 in the situation where the cartridge 100 is not properly interfaced with the optical imaging system 200. Since the transducers 186 may be sensitive to time of flight, the system may be able to reconstruct and collect depth scanning. A physical transducer 186 can be integrated within the cartridge (either on the container walls or optical substrate 120). The transducer 186 would have physical electrical contacts that interface to a connector. FIG. 1D shows three options for location of transducer 186 and subsequent electrical vias 188. Electrical vias 188 can also be integrated into the optical substrate 120 or on walls of cartridge to increase distance from location of actual transducer 186. A liquid can be added to the cartridge to allow improved acoustic wave propagation.

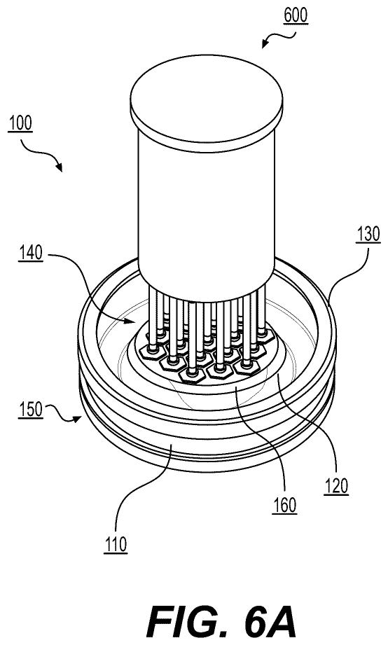

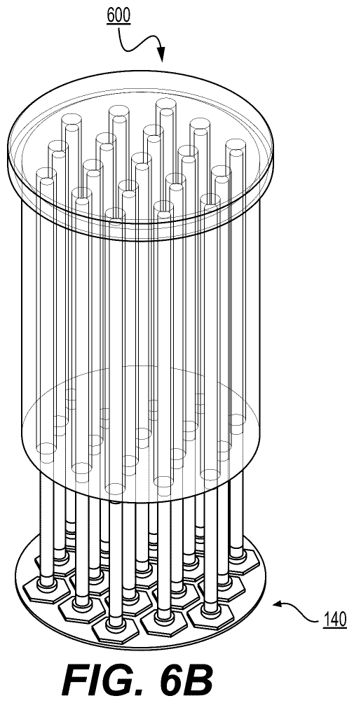

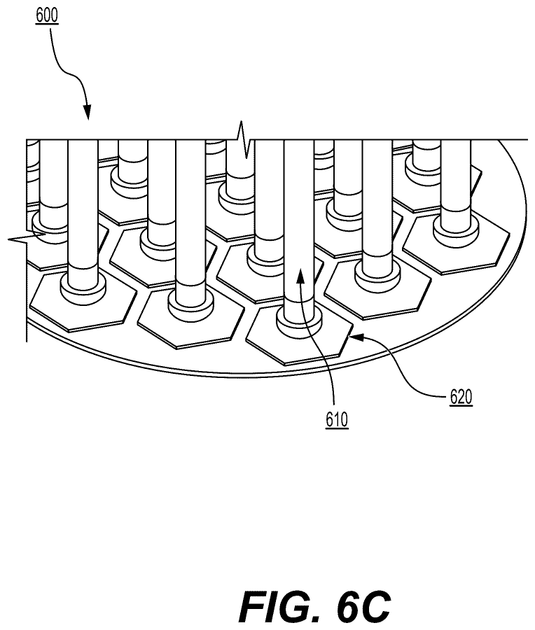

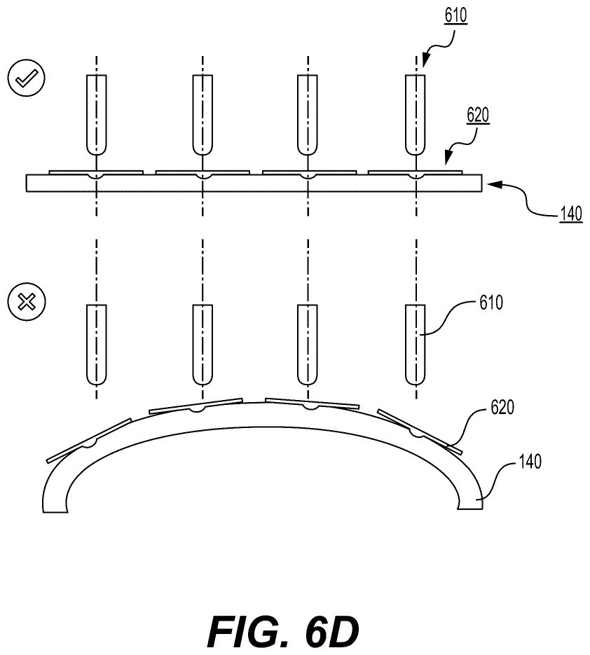

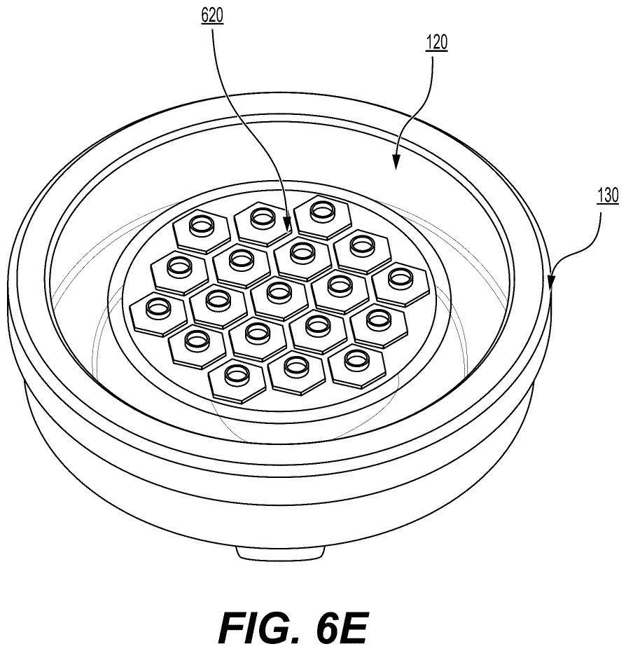

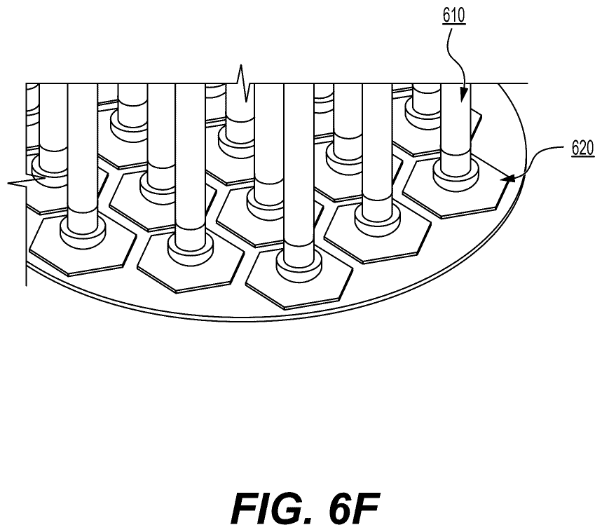

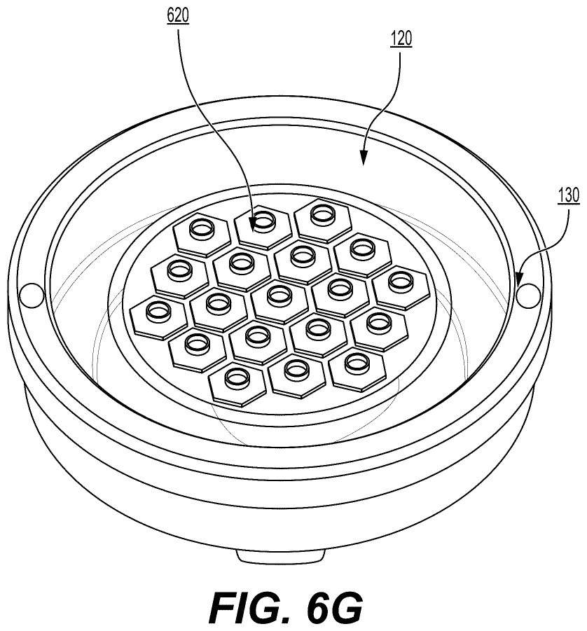

The lid 130 may be provided on a top surface of the container 110, and may include a membrane 140 to compress the un-labelled tissue 160 against the optical substrate 120 such that an entire margin 170 of the un-labelled tissue 160 is flat against the optical substrate 120. Herein, “flat” may refer to a surface of the un-labelled tissue 160 being in contact with a surface of the optical substrate 120 (e.g., the top surface 120-1) such that no gap exists between the surface of the un-labelled tissue 160 and the surface of the optical substrate 120. For example, as shown in FIG. 1, the membrane 140 may receive external pressure 105 (i.e., from outside of cartridge 100), and transmit internal pressure 115 (from within cartridge 100) to the un-labelled tissue 160 to compress the un-labelled tissue 160 against the optical substrate 120. The membrane 140 may receive external pressure 105 from a human operator, from a pressure assembly 600 (as described in more detail in connection with FIG. 6), or the like. Based on pressure being applied, the membrane 140 may conform to the surface of the un-labelled tissue 160 and may compress the un-labelled tissue 160 against the optical substrate 120. The lid 130 may be formed of any suitable material, and may be any suitable shape. Further, the membrane 140 may be formed of any suitable material, and may be any suitable shape. The external pressure 105 could be used in conjunction with an internal vacuum. e.g., a vacuum is pulled to remove air pockets and residual unwanted fluid. The vacuum may not be sufficient enough to make tissue flat, so an external pressure 105 can be used after. Alternatively, an external pressure 105 (like a repeating thumping) can be used to displace fixed air bubbles and the vacuum can be used to pull those air bubbles out.

The cap 150 may be provided on a bottom surface of the container 110, and may protect the optical substrate 120. The cap 150 may be formed of any suitable material, and may be any suitable shape. The optical imaging system 200 may automatically remove the cap 150 by a mechanical action such that the optical substrate 120 is not inadvertently damaged during installation of the un-labelled tissue 160 into the cartridge 100. The cap 150 may be referred to as a “bottom cap” such as in situation where a “top cap” (not shown) is provided over the lid 130 (e.g., in the case where the membrane is permeable). The “top cap” would seal the unit for proper storage of fresh tissue. If the membrane is completely impermeable to fluid transfer, then the top cap might not be needed.

A surgeon, medical professional, or other entity may resect the un-labelled tissue 160 from a patient, and place the un-labelled tissue 160 on the optical substrate 120 in the container 110. As examples, the un-labelled tissue 160 may be rinsed before being placed in the container 110, may be fixed before being placed in the container 110, or may be directly placed in the container 110 without any processing (for example, immediately after resection without any intervening processing steps between resection and placement into the container). In some embodiments, the un-labelled tissue 160 may be placed in the cartridge within a threshold time frame of being resected from the patient (e.g., within one minute, within five minutes, within ten minutes, etc.).

In some implementations, a fluid may be added into the cartridge 100 to aid in imaging. As examples, the fluid may be saline, water, methanol, ethanol, acetic acid, acetic acid and ethanol, formaldehyde, paraformaldehyde, picrates, hepes-glutamic acid buffer-mediated organic solvent, aluminum chloride, or the like. Additionally, or alternatively, fixation mediums (e.g., formalin, paraffin, etc.) may be added (e.g., pumped in via a fluid port 185) to the cartridge 100 to preserve the un-labelled tissue 160 during storage of the un-labelled tissue 160 after imaging of the un-labelled tissue 160.

As shown in FIG. 1E, fluid ports 185 may be provided on a top position 190. Fluid can be added or removed via the fluid port(s) 185. When vacuum is applied to fluid port(s) 185 the flexible membrane 140 will be compressed and will conform to the tissue 160 and tissue 160 will be compressed. In some implementations, a fluid channel 188 may fluidly connect the fluid ports 185 to container 110.

As shown in FIG. 1F, fluid ports 185 may be provided on a bottom position. In this case, the fluid ports 185 are on the bottom side 192. The ports 185 could engage in quick disconnect fittings on the machine side. In this way, vacuum can be pulled and/or liquids can be added from the bottom/machine side which cleans up the user side (no fluid or tubing obstruction for user). Check valves can be added to only allow one way flow as desired.

As shown in FIG. 1G, fluid ports 185 may be provided on a top position 190. When no vacuum is applied, the membrane 140 is not deformed. When vacuum is applied to port(s), the membrane 140 is deformed to tissue 160. Membrane can either be plastically or elastically deformed.

As shown in FIG. 1H, an external pressure 105 can be applied to the back/top side of the tissue (through the membrane 140) which serves two functions: 1) Add extra pressure to promote contact of tissue/optical substrate 120, and 2) Add pressure in such a way that promotes air bubbles 194 leaving the tissue/glass interface. The combination of vacuum also helps air bubbles 194 escape. The pressure can be static, or alternating (“repeated thumping”) to promote air bubbles 194 to leave. In both cases, the overview camera (210) can be used to identify where pressure needs to be added so as to move the air bubble 194 from the optical substrate-tissue interface 120. Instead of the overview camera, the optical imaging head (220) can be activated to see the air bubble 194. For example, a scattering image using detection wavelength will clearly show presence of air bubble 194.

As shown in FIG. 1I, an external pressure array 107 can also be used in conjunction with the vacuum. For example, the external pressure array 107 may be a spring loaded actuator, a pogo-pin array, or the like. In the case of the pogo-pin array, the pins can be individually activated so as to chase air bubbles 194 out of the optical substrate-tissue interface 120 or add pressure/force over certain areas only.

As shown in FIG. 1J, in the uncompressed state, deep margin 163 of the resected tissue 196 is against the optical substrate 120 and the peripheral margin 164 is raised. The complete margin can be mapped out based on the standard anatomical position. The resected tissue 196 may be displayed from a top view, side view, and bottom view (as displayed from left to right in FIG. 1J, FIG. 1K, and FIG. 1L). The resected tissue may include visible cancer 197.

As shown in FIG. 1K, pressure is first applied to the left side so that margin section 1 161 is completely flat against the optical substrate and in focus. Margin section 1 161 is the first scanning area.

As shown in FIG. 1L, upon completion of the first scan, pressure is then applied to the right side so that margin section 2 162 is completely flat against the optical substrate and in focus. Margin section 2 162 is the second scanning area.

As shown in FIG. 1M, it is desirable that the optical substrate 120 is flat along the z=0 plane, such that the tissue interface remains at the focus on the optical beams for ideal imaging. When pressure is applied or when vacuum is applied, the optical substrate 120 (and tissue interface) is deformed so that the image would be out of focus. By knowing the discrete value of pressure or vacuum applied, we can pre determine the “bending” profile 198 of the optical substrate 120 and during scanning we can move the tissue sample up/down along the profile 198 of the bending such that the image remains in focus. Alternatively the combination of pressure and vacuum can ‘offset’ each other such that the optical substrate and tissue remains flat.

FIG. 2 is a diagram of a cartridge interfacing with an optical imaging system. As shown in FIG. 2, the cartridge 100 may interface with the optical imaging system 200. For example, as shown, the cartridge 100 may interface with a cartridge plate 230 of the optical imaging system 200. The optical imaging system 200 may include, among other things, a camera head 210, an imaging head 220, and the cartridge plate 230. The optical imaging system 200 may be configured to move the cartridge plate 230 such that the cartridge 100 is disposed above the camera head 210 or the imaging head 220 to permit imaging of the un-labelled tissue 160. As further shown in FIG. 2, the optical imaging system 200 may be configured to generate a virtually-stained histological image 240 of the un-labelled tissue 160. Additionally, or alternatively, the optical imaging system 200 may be configured to generate other types of images of the un-labelled tissue 160.

In some implementations, the optical imaging system 200 may be a photoacoustic remote sensing imaging system, such as photon absorption remote sensing imaging system. In this case, the optical imaging system 200 may use a picosecond scale pulsed excitation laser 176 (as shown in FIG. 1B) that is focused into the un-labelled tissue 160 to generate radiative effects (e.g., optical emissions), non-radiative effects (e.g., heat and pressure), and scattering effects in the un-labelled tissue 160. Further, the optical imaging system 200 may capture and convert photons into different forms of emission from the un-labelled tissue 160 (e.g., non-radiative and radiative) while scattered photons continue moving through and interacting with other portions of the un-labelled tissue 160. Further still, the optical imaging system 200 may be a photoacoustic remote sensing imaging system, such as a photo-thermal imaging system. In this case, the optical imaging system 200 may record non-radiative effects using a secondary confocal detection beam, thereby enabling the detection of temperature or pressure changes. The optical imaging system 200 may register the changes as modulations in backscattering intensity, and directly correlate the modulations to the local non-radiative absorption contrast. The unperturbed backscatter (pre-excitation event) simultaneously captures the optical scattering contrast. In this way, the optical imaging system 200 may combine captured contrasts or visualize the captured contrasts separately. The optical imaging system 200 may be configured to implement one or more techniques as described in U.S. Pat. No. 10,117,583 issued on Nov. 6, 2018; U.S. Pat. No. 10,327,646 issued on Jun. 25, 2019; U.S. Pat. No. 10,627,338 issued on Apr. 21, 2020; U.S. Publication No. 2020/0359903 published on Nov. 19, 2020; U.S. Publication No. 2021/0199566 published on Jul. 1, 2021; U.S. Publication No. 2021/0404948 published on Dec. 30, 2021; U.S. Pat. No. 11,122,978 issued on Sep. 21, 2021; International PCT Publication No. WO 2021/255695 published on Dec. 23, 2021; and PCT Application No. PCT/IB2022/054433 filed on May 12, 2022, which are hereby incorporated by reference in their entireties.

To generate the virtually-stained histological image 240 of the un-labelled tissue 160, the optical imaging system 200 may use ultraviolet light to virtually-stain the un-labelled tissue 160, and then color match the virtually-stained un-labelled tissue 160 to hematoxylin and eosin stains. In this way, the optical imaging system 200 can intraoperatively generate substantially similar histological images as compared to the time-intensive and error-prone tissue processing and staining workflow as addressed above. The virtually-stained histological image 240 may be an image of the un-labelled tissue 160 including emulated hematoxylin stains which stain cell nuclei with a deep blue-purple color, and emulated eosin stains which stain cytoplasm and extracellular matrix with pink shades.

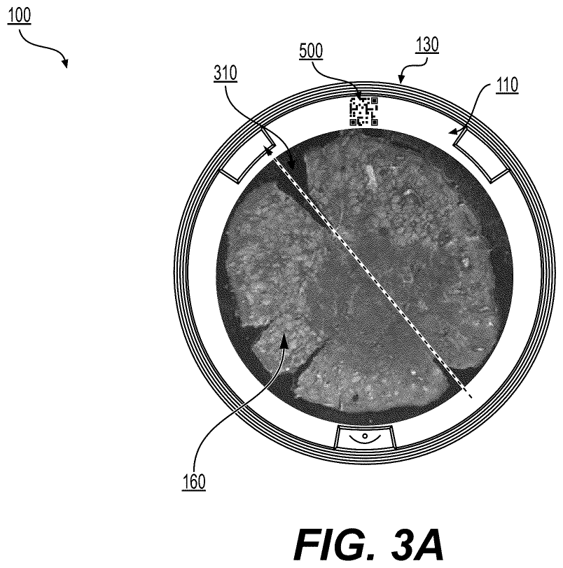

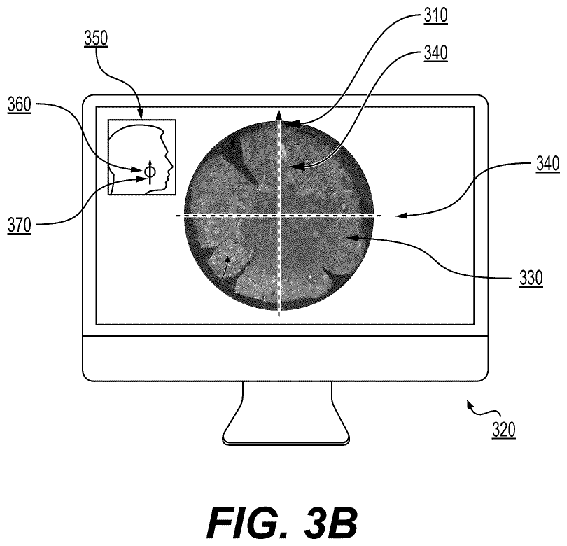

FIG. 3A is a diagram of a cartridge storing an un-labelled tissue including a notch. As shown in FIG. 3A, the un-labelled tissue 160 may include a notch 310. The notch 310 may be a formation in the un-labelled tissue 160 that allows maintenance of an orientation of the un-labelled tissue 160 relative to the patient. For example, as shown in FIG. 3B, the notch 310 may correspond to a vertical (superior to inferior) direction of the patient. In other examples, the notch may correspond to horizontal directions within the patient, such as, e.g., a lateral-medial direction, or anterior-posterior direction. After excising the un-labelled tissue 160 from the patient, the notch 310 may be applied to, cut into, or otherwise formed into the un-labelled tissue 160, and the un-labelled tissue 160 then may be placed in the cartridge 100. Because the un-labelled tissue 160 is not processed as compared to other techniques, the notch 310 is at less risk of being lost or damaged

FIG. 3B is a diagram of a display including a tissue image and a patient orientation image. As shown in FIG. 3B, the display 320 may display a tissue image 330, tissue image axes 340, a patient image 350, a tissue image 360, and a direction indicator 370.

The tissue image 330 may be an image of the un-labelled tissue 160 as imaged by the optical imaging system 200. The optical imaging system 200 may image the un-labelled tissue 160, detect the notch 310, and cause the display 320 to display the un-labelled tissue 160 in an orientation that corresponds to an orientation of the un-labelled tissue 160 relative to the patient, based on the notch. For example, as shown in FIG. 3B, the notch 310 may correspond to a vertical direction of the patient. Accordingly, as shown, the display 320 may display the tissue image 330 such that the notch 310 is disposed in the vertical direction.

The tissue image 330 may include tissue image axes 340 overlaid on the tissue image 330. The tissue image axes 340 may include a vertical axis (superior-inferior axis) and a horizontal axis (lateral-medial axis), as shown. The optical imaging system 200 may cause the tissue image axes 340 to correspond to the notch 310. That is, as shown, the vertical axis of the tissue image axes 340 may be planar with a direction indicated by the notch 310. For example, as shown in FIG. 3B, the notch 310 may correspond to a vertical direction of the patient. Accordingly, as shown, the display 320 may display the tissue image axes 340 on the tissue image 330 such that the vertical axis of the tissue image axes 340 aligns with the notch 310.

The patient image 350 may be a representation of a body part of the patient, and may include a tissue image 360 overlaid on the representation of the body part at a corresponding position at which the un-labelled tissue 160 was resected from the patient. For example, as shown in FIG. 3B, the patient image 350 includes a tissue image 360 provided on a right cheek of the patient.

The optical imaging system 200 may receive information that identifies the location at which the un-labelled tissue 160 was resected from the patient, and generate the patient image 350 and the tissue image 360 based on the information that identifies the location. For example, the optical imaging system 200 may receive the information based on an image captured by the optical imaging system 200, based on a user input, based on information stored on the cartridge 100, based on an artificial intelligence (AI) technique, based on analyzing the un-labelled tissue 160, or the like. Further, the optical imaging system 200 may generate the patient image 350 such that the tissue image 360 is overlaid on the patient image 350 at a location corresponding to the location at which the un-labelled tissue 160 was resected from the patient. Traditional workflows use inks to indicate orientation of sample. Nominally, those inks are not good for PARS since the ink absorbs our excitation and/or detection wavelengths. In some implementations, an ink with an absorption spectrum outside of the excitation and/or detection wavelengths of interest so the inks do not interfere with the optical measurement. In some implementations, the surgeon may use an ink that is PARS specific. In this way, surgeons are not required to change their current inking practice.

The direction indicator 370 may be an indicator that depicts an orientation of the un-labelled tissue 160 relative to the patient, as determined by the notch 310. For example, as shown in FIG. 3B, the optical imaging system 200 may overlay the direction indicator 370 which indicates a vertical direction on the tissue image 360 and the patient image 350.

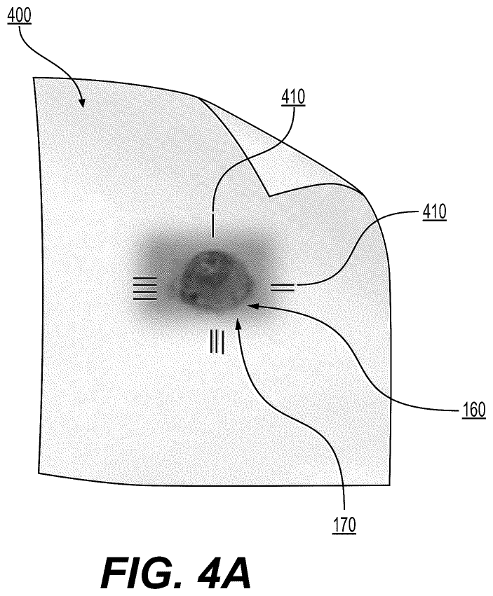

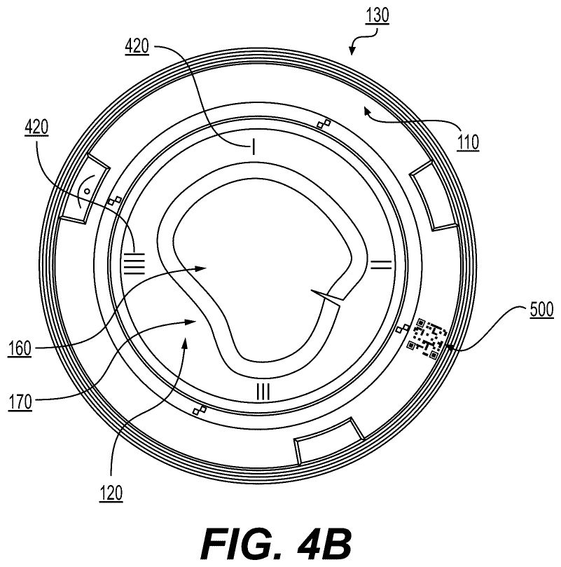

FIG. 4A is a diagram of a paper including orientation marks. In addition to the function of orientation marks, the paper also serves the functions of a blotting cloth and a cutting surface. As shown in FIG. 4, a paper 400 may include orientation marks 410. The paper 400 may be paper on which the un-labelled tissue 160 is placed after being resected from the patient and before being placed in the cartridge 100. The orientation marks 410 may be marks that permit an orientation of the un-labelled tissue 160 relative to the patient to be maintained. For example, as shown in FIG. 4A, orientation marks 410 depicted as “I” and “III” may correspond to a vertical direction of the patient (e.g., inferior-superior), and the orientation marks 410 depicted as “IIII” and “II” may correspond to a horizontal direction of the patient (e.g., anterior-posterior or lateral-medial). The un-labelled tissue 160 is placed on the paper 400 and oriented with respect to the orientation marks 410 such that the orientation of the un-labelled tissue 160 with respect to the patient can be ascertained.

FIG. 4B is a diagram of a cartridge including orientation marks. As shown in FIG. 4B, the optical substrate 120 may include orientation marks 420 that permit maintenance of an orientation of the un-labelled tissue 160 relative to the patient. For example, the orientation marks 420 depicted as “I” and “III” may correspond to a vertical direction of the patient (e.g., inferior-superior), and the orientation marks 420 depicted as “IIII” and “II” may correspond to a horizontal direction of the patient (e.g., anterior-posterior or lateral-medial). The paper 400 may include the orientation marks 410 that correspond to the orientation marks 420 provided on the optical substrate 120. In particular, the paper 400 may have the exact same markings in the exact same orientation as the orientation marks 420 on optical substrate 120. The un-labelled tissue 160 may then be transferred, by the same person or entity that performed the resection, or by a different person or entity, to the optical substrate 120 while maintaining an orientation of the un-labelled tissue 160 relative to the orientation marks 420. For example, the resected un-labelled tissue 160 may be placed by a surgeon on to the paper 400 in an intended orientation relative to the orientation marks 410 of the paper 400, and then an assistant, technician, nurse, other physician, for example, may transfer the un-labelled tissue 160 to the optical substrate 120 by visually aligning the markers of the paper 400 and the orientation marks 420 of the cartridge 100 in order to keep the orientation intended by the surgeon.

In this way, the optical imaging system 200 may detect the orientation marks 420, and cause a display 320 to display the un-labelled tissue 160 in an orientation that corresponds to an orientation of the un-labelled tissue 160 relative to the patient. Additionally, the optical imaging system 200 may superimpose the orientation marks on the image displayed by the display 320. Although the orientation marks 420 are depicted as being provided on the optical substrate 120, the orientation marks 420 may be provided on any of the other components of the cartridge 100, such as the lid 130, the container 110, or the cap 150. For example, the cap 150 may be in place when the un-labelled tissue 160 is placed into the cartridge 100, and might have orientation marks 420 that are large and visible to an un-aided human eye and that match the orientation marks 420 on the paper 400. If the cap 150 is keyed to the cartridge 100, alignment of the un-labelled tissue 160 relative to the cartridge 100 may be maintained after the cap 150 is removed.

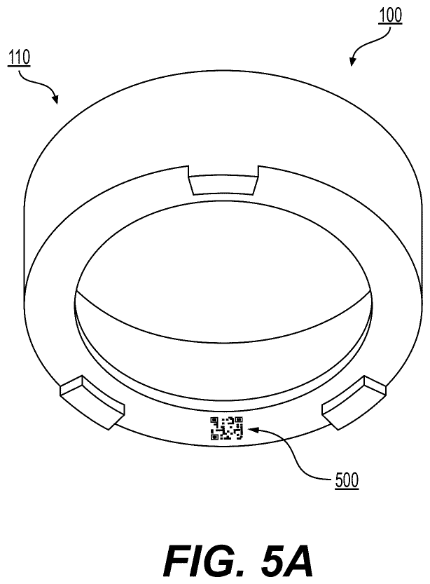

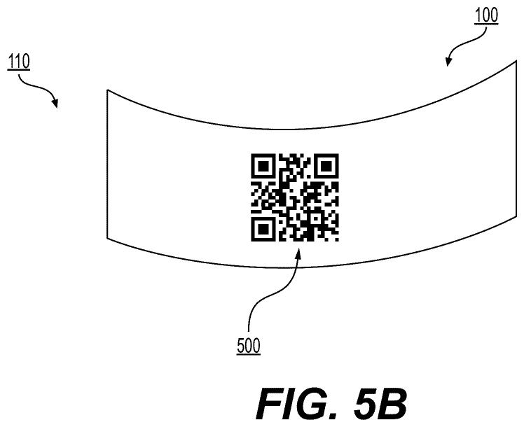

FIGS. 5A and 5B are diagrams of a cartridge including a unique identifier. As shown in FIGS. 5A and 5B, the container 110 may include a unique identifier 500. The unique identifier 500 may be an identifier that can be electronically tracked. For example, the unique identifier 500 may be electronically tracked by the optical imaging system 200 via a communication technique such as via quick response (QR) code, radio frequency identification (RFID), near field communication (NFC), Bluetooth, bar code, or the like. As an example, based on the container 110 being interfaced with the optical imaging system 200, the optical imaging system 200 (e.g., the camera head 210) may read the unique identifier 500 provided on a bottom surface of the container 110 and obtain the unique identifier 500. It is also possible to use the detection laser as a scattering microscope which could also image the barcode (2d or 1d). In this case a visible camera may not be needed. Although FIGS. 5A and 5B depict the unique identifier 500 as being provided in the form of a QR code provided on a bottom surface of the container 110, the unique identifier 500 may be provided at any position on any of the other components of the cartridge 100, such as the lid 130, the optical substrate 120, or the cap 150. The unique identifier 500 may be correlated with a patient name, a patient identifier, a tissue identifier, or the like.

Additionally, or alternatively, the unique identifier 500 may be an interlock for operation of the optical imaging system 200. For example, operation of the imaging head 220 of the optical imaging system 200 may be prevented until the cartridge 100 is interfaced with the optical imaging system 200 and the unique identifier 500 is detected by the optical imaging system 200. Alternatively, the cartridge 100 may include another type of interlock provided in a different position or on another component of the cartridge 100. For example, the cartridge 100 may include a mechanical feature that engages with the optical imaging system 200 when the cartridge 100 interfaces with the optical imaging system 200.

Additionally, or alternatively, the unique identifier 500 may be a control mechanism that prevents the cartridge 100 from being used multiple times. For example, the optical imaging system 200 may detect the unique identifier 500, and determine whether the cartridge 100 has already been used for imaging. Based on determining that the cartridge 100 has already been used for imaging, the optical imaging system 200 may prevent additional imaging of the un-labelled tissue 160 provided in the cartridge 100. In this way, the unique identifier 500 may prevent, or reduce, a number of false positives or false negatives that occur based on cross-contamination.

Additionally, or alternatively, the unique identifier 500 may include information that permits the optical imaging system 200 to provide an appropriate amount of pressure or displacement to the cartridge 100 to compress the un-labelled tissue 160 against the optical substrate 120 such that the entire margin of the un-labelled tissue 160 is flat against the optical substrate 120. For example, the unique identifier 500 may include information identifying a pressure value to apply to the cartridge 100, a displacement value for the cartridge 100, an AI algorithm to be executed by the optical imaging system 200 to apply pressure to the cartridge 100, or the like. The identifier may also associate the country being marketed to. The identifier also could associate regulatory approval vs IDE or RUO.

FIG. 6A is a diagram of a cartridge interfacing with a pressure assembly of an optical imaging system. FIG. 6B is a diagram of a pressure assembly of an optical imaging system interfacing with a membrane of a cartridge. FIG. 6C is a diagram of pins and pads of a pressure assembly of an optical imaging system. As shown in FIGS. 6A-6C, a pressure assembly 600 may be configured to apply pressure to the un-labelled tissue 160 to compress the un-labelled tissue 160 against the optical substrate 120 such that an entire margin 170 of the un-labelled tissue 160 is flat against the optical substrate 120. In some implementations, the pressure assembly 600 may be external to the cartridge 100. For example, the optical imaging system 200 may include the pressure assembly 600. Alternatively, the pressure assembly 600 may be internal to the cartridge 100. The individual actuators in the pressure assembly can be individually addressed so as to actuate only one at a time (or a combination of multiples at the same time).

As shown in FIGS. 6A-6C, and as an example, the pressure assembly 600 may include an array of pins 610 and corresponding pads 620. By usage of the pins 610 and the pads 620, the pressure assembly 600 may exert different pressures at different displacements (different locations along the surface of membrane 140 and/or lid 130). For example, the pads 620 may collectively cover an entire surface, a substantial entirety, or another portion, of the un-labelled tissue 160. Further, each pad 620 may individually cover a subset of the surface of the un-labelled tissue 160. Further still, the pressure applied to each pad 620 via respective pins 610 may be adjusted and varied such that a same, or different, pressure can be applied to each pad 620. In this way, the pressure applied to the surface of the un-labelled tissue 160 may be consistent across the entire surface of the un-labelled tissue 160 (e.g., where each pad 620 applies a same pressure), or may vary across the surface of the un-labelled tissue 160 (e.g., where one or more pads 620 applies different pressures). In this way, the pressure assembly 600 may compress the un-labelled tissue 160 such that the entire margin 170 of the un-labelled tissue 160 is flat against the optical substrate 120.

In some implementations, and as shown in FIGS. 1J-1L, the optical imaging system 200 and/or the cartridge 100 may be configured to apply a positive pressure on the bottom surface of the optical substrate 120 to counteract bending of the optical substrate 120, and to maintain the flatness of the entire margin 170 of the un-labelled tissue 160 against the optical substrate 120.

The optical imaging system 200 may be configured to control the pressure assembly 600 to modify an applied pressure such that the entire margin 170 of the un-labelled tissue 160 is flat against the optical substrate 120. In some implementations, the optical imaging system 200 may control the pressure assembly 600 to apply a particular pressure based on information obtained from the unique identifier 500 of the cartridge 100. For example, the unique identifier 500 may include pressure information identifying the particular pressure to apply based on the particular type of un-labelled tissue 160 to be imaged. The pressure information may include information identifying a particular pressure to be applied by each pin 610 and pad 620 group of the pressure assembly 600. For example, the pressure information may include a matrix of pressures to be applied to the un-labelled tissue 160 that corresponds to the groups of pins 610 and pads 620. In other words, the pressure information may identify a particular pressure to be applied by each pad 620 of the pressure assembly 600, which corresponds to a particular subset of a surface of the un-labelled tissue 160. The pressure information may be pre-determined, may be determined using an AI technique, or the like.

Additionally, or alternatively, the optical imaging system 200 may modify an applied pressure based on images of the un-labelled tissue 160. For example, the optical imaging system 200 (e.g., the camera head 210) may image the un-labelled tissue 160, and the optical imaging system 200 may control the pressure assembly 600 to modify an applied pressure such that the entire margin 170 of the un-labelled tissue 160 is flat against the optical substrate. In some implementations, an AI algorithm executed by the optical imaging system 200 may control the pressure assembly 600 to perform the foregoing operations.

As shown in FIG. 6D, before Pressure Assembly 600 contacts the membrane, the Pad array location and orientation might already match the Pin array 610. That is, the Pins 610 engage with the Pads 620 before the Membrane is stretched.

As shown in FIG. 6E, the Pad array 620 is attached to the Membrane, not the Pins. In this way, there is no need for tiny permanent spheroid joint on Pressure Assembly 600. Further, the Pad array 620 could potentially hold its shape after pressure is applied, which will hold the tissue in the current orientation even after the Pressure Assembly 600 disengages.

As shown in FIG. 6F, each Pad 620 contains a cup-like depression and each Pin 610 contains a rounded tip, which forms a spheroid joint upon contact.

As shown in FIG. 6G, the volume inside the Cartridge is sealed. A vent hole may be needed to divert air and fluids. Another hole can be used for adding fixative or embedding medium. The ports can be built into the Pressure Assembly 600, which will be connected when it engages with the Lid 130.

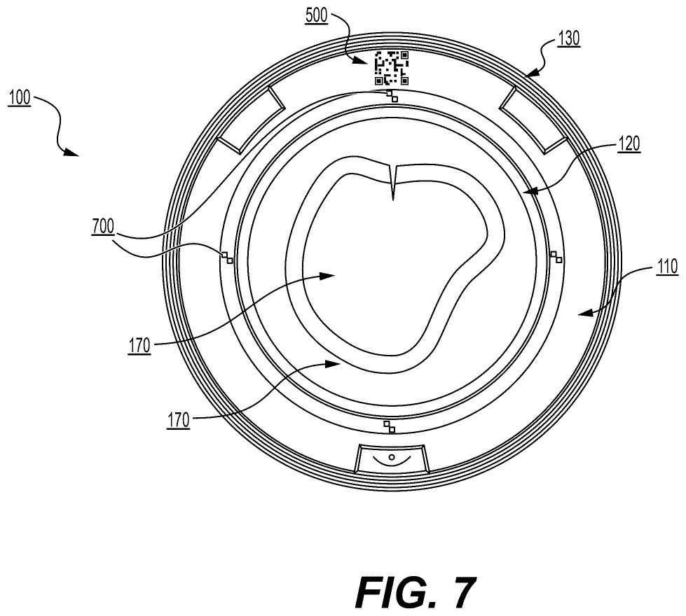

FIG. 7 is a diagram of a cartridge including fiducials. As shown in FIG. 7, the optical substrate 120 may include fiducials 700 that aid in the alignment between the camera head 210 and the imaging head 220 of the optical imaging system 200 to ensure that PARS acquisition is centered about an appropriate interrogation window. The fiducials 700 may either be laser etched on the optical substrate 120 or deposited on the optical substrate 120 using photolithography tools (e.g., thin metal that is deposited and etched on a wafer or die level). The fiducials 700 may be outside of the un-labelled tissue 160 imaging window (e.g., an outer annular ring of the optical substrate 120) so as to not impede PARS imaging of the tissue, and allow a high laser density to be used without damaging the optical substrate 120. The fiducials may be the right shape/size such that the visible camera has enough optical resolution to resolve. In some implementations, at least 3 fiducials are needed to establish x,y orientation. When fiducials are scanned by the PARS head, the fiducials should be within the optical field of view of the imaging system.

In some implementations, the fiducials 700 may be optical resolution targets that aid in both axial auto alignment (focusing direction) and lateral alignment of the detection and excitation beams of the optical imaging system 200. By measuring the fiducials in all four quadrants, the optical imaging system 200 may auto level the cartridge plate 230 relative to the beam to ensure the focal plane is consistent across the entire field of view of the un-labelled tissue 160 during a scanning acquisition.

By using scattering images from both excitation and detection lasers, the fiducials 700 may be optically resolved, and, if done at the start of each new PARS acquisition, the fiducial image quality can act as a beam health metric to ensure that the excitation and detection beams stay co-focused over time to avoid system drift. If the scattering image of either excitation or detection falls out or focus, the optical imaging system 200 may trigger an alarm that indicates that the optical imaging system 200 needs realignment.

In some implementations, if an active alignment system is implemented by the optical imaging system 200, the scattering images of the fiducials 700 can be used to manipulate the beam(s) in order to re-gain alignment of the optical imaging system 200.

In light of the foregoing, during surgery, tissue may be resected from a patient, and placed in the cartridge 100. The cartridge 100 then may be interfaced with the optical imaging system 200. The optical imaging system 200 may intraoperatively generate a virtually-stained histological image 240 of a true margin 170 of the un-labelled tissue 160 while the cartridge 100 maintains a tissue orientation as resected from the patient. Moreover, in this way, the embodiments herein permit the ascertaining of whether a negative margin exists in a more accurate manner, in a quick manner, and intraoperatively, which thereby improves the safety and efficacy of surgical excision of cancerous tumors.

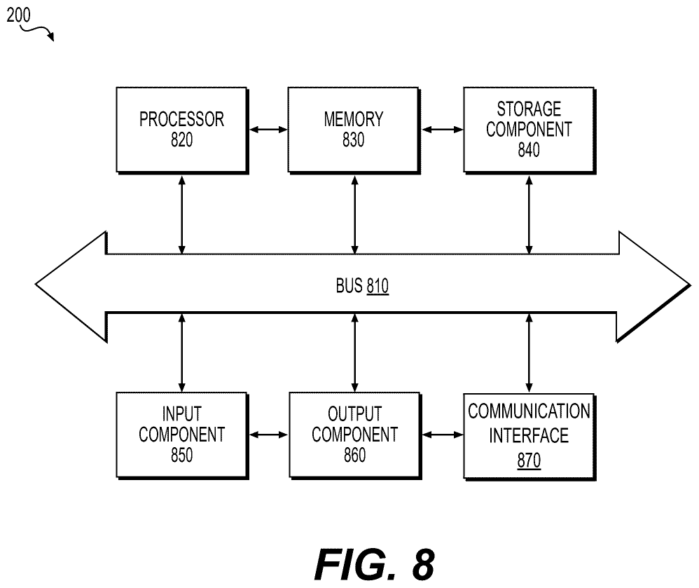

FIG. 8 is a diagram of components of an optical imaging system. As shown in FIG. 8, the optical imaging system 200 may include a bus 810, a processor 820, a memory 830, a storage component 840, an input component 850, an output component 860, and a communication interface 870. The optical imaging system 200 may include the foregoing components in addition to the components described in connection with FIGS. 2 and 6A-6C (e.g., the camera head 210, the imaging head 220, and the pressure assembly 600).

The bus 810 includes a component that permits communication among the components of the optical imaging system 200. The processor 820 may be implemented in hardware, firmware, or a combination of hardware and software. The processor 820 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component.

The processor 820 may include one or more processors capable of being programmed to perform a function. The memory 830 may include a random access memory (RAM), a read only memory (ROM), and/or another type of dynamic or static storage device (e.g., a flash memory, a magnetic memory, and/or an optical memory) that stores information and/or instructions for use by the processor 820. The storage component 840 may store information and/or software related to the operation and use of the optical imaging system 200. For example, the storage component 840 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

The input component 850 may include a component that permits the optical imaging system 200 to receive information, such as via user input (e.g., a touch screen display, a keyboard, a keypad, a mouse, a button, a switch, and/or a microphone for receiving the reference sound input). Additionally, or alternatively, the input component 850 may include a sensor for sensing information (e.g., a global positioning system (GPS) component, an accelerometer, a gyroscope, and/or an actuator). The output component 860 may include a component that provides output information from the optical imaging system 200 (e.g., a display 320, a speaker for outputting sound at the output sound level, and/or one or more light-emitting diodes (LEDs)).

The communication interface 870 may include a transceiver-like component (e.g., a transceiver and/or a separate receiver and transmitter) that enables the optical imaging system 200 to communicate with other devices, such as via a wired connection, a wireless connection, or a combination of wired and wireless connections. The communication interface 870 may permit the optical imaging system 200 to receive information from another device and/or provide information to another device. For example, the communication interface 870 may include an Ethernet interface, an optical interface, a coaxial interface, an infrared interface, a radio frequency (RF) interface, a universal serial bus (USB) interface, a Wi-Fi interface, a cellular network interface, or the like.

The optical imaging system 200 may perform one or more processes described herein. The optical imaging system 200 may perform these processes based on the processor 820 executing software instructions stored by a non-transitory computer-readable medium, such as the memory 830 and/or the storage component 840. A computer-readable medium is defined herein as a non-transitory memory device. A memory device may include memory space within a single physical storage device or memory space spread across multiple physical storage devices. The software instructions may be read into the memory 830 and/or the storage component 840 from another computer-readable medium or from another device via the communication interface 870. When executed, the software instructions stored in the memory 830 and/or the storage component 840 may cause the processor 820 to perform one or more processes described herein.

Additionally, or alternatively, hardwired circuitry may be used in place of or in combination with software instructions to perform one or more processes described herein. Thus, implementations described herein are not limited to any specific combination of hardware circuitry and software.

The number and arrangement of the components shown in FIG. 8 are provided as an example. In practice, the optical imaging system 200 may include additional components, fewer components, different components, or differently arranged components than those shown in FIG. 8. Additionally, or alternatively, a set of components (e.g., one or more components) of the optical imaging system 200 may perform one or more functions described as being performed by another set of components of the optical imaging system 200.

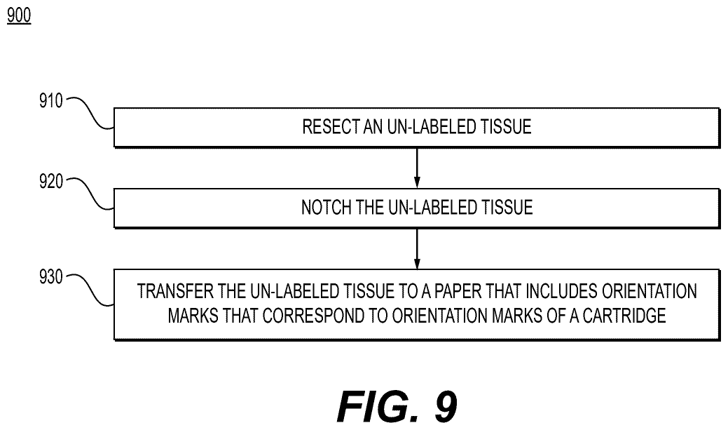

FIG. 9 is a flowchart of a process for preparing an un-labelled tissue for imaging. As shown in FIG. 9, a process 900 for preparing an un-labelled tissue 160 for imaging may include resecting the un-labelled tissue 160 from a patient (operation 910), notching the un-labelled tissue 160 (operation 920), and transferring the un-labelled tissue 160 to a paper 400 that include orientation marks 410 that correspond to orientation marks 420 of a cartridge 100 (operation 930). For example, a surgeon, or other medical personnel, may resect the un-labelled tissue 160 from the patient, notch the un-labelled tissue 160, and place the un-labelled tissue 160 on the paper 400. In some cases, the process 900 may be performed within a threshold time frame (e.g., one minute, five minutes, ten minutes, etc.). Alternatively, the notch can also be done during the actual resection (i.e., the surgeon, or medical personnel, can make a notch while the tissue is still on/in the patient).

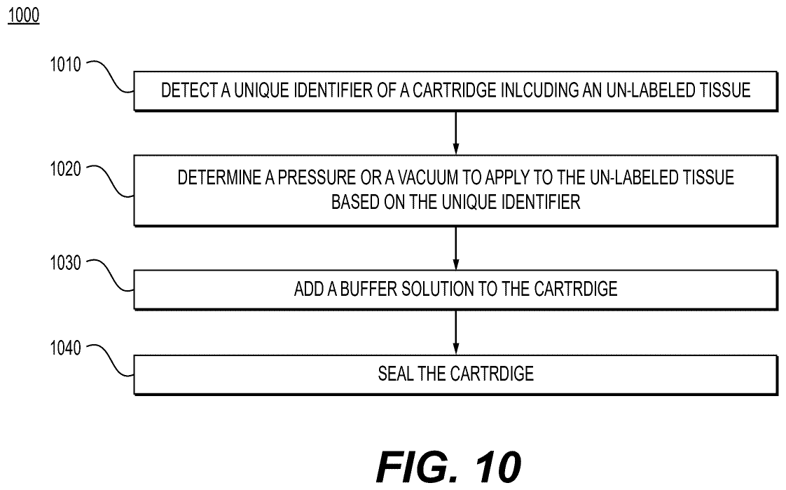

FIG. 10 is a flowchart of a process for preparing a cartridge for imaging. As shown in FIG. 10, a process 1000 may include obtaining a unique identifier 500 of a cartridge 100 including an un-labelled tissue 160 (operation 1010). For example, the optical imaging system 200 may detect the unique identifier 500, such as by reading a serial number, via RFID, via QR, or the like. As further shown in FIG. 10, the process 1000 may include determining a pressure or a vacuum to apply to the un-labelled tissue 160 based on the unique identifier 500 (operation 1020). For example, the optical imaging system 200 may determine pressure or vacuum information, such as an internal pressure, an external pressure, a compliant compression setting, a vacuum setting, a pressure assembly 600 pressure, a positive pressure, or the like. As a particular example, the optical imaging system 200 may determine information identifying pressure or vacuum to be applied by each respective pin 610 and pad 620 group of the pressure assembly 600. As further shown in FIG. 10, the process 1000 may include adding a buffer solution to the cartridge 100 (operation 1030) and sealing the cartridge 100 (operation 1040).

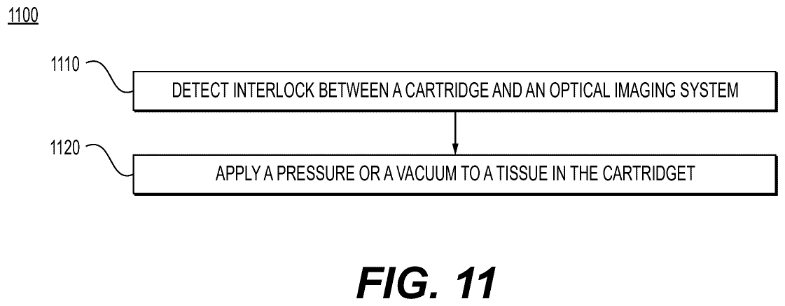

FIG. 11 is a flowchart of a process for detecting an interlock between a cartridge and an optical imaging system. As shown in FIG. 11, a process 1100 may include detecting an interlock between a cartridge 100 and an optical imaging system 200 (operation 1110). For example, the optical imaging system 200 may detect a mechanical feature that engages with the optical imaging system 200 when the cartridge 100 interfaces with the optical imaging system 200, and detect the interlock based on detecting the mechanical feature. Alternatively, the optical imaging system 200 may optically detect the cartridge 100, such as via the camera head 210, and detect the interlock based on optically detecting the cartridge 100. Additionally, or alternatively, the optical imaging system 200 may detect a unique identifier 500 of the cartridge 100, and detect the interlock based on optically detecting the unique identifier 500 of the cartridge 100.

As further shown in FIG. 11, the process 1100 may include applying a pressure or a vacuum to an un-labelled tissue in the cartridge 100 (operation 1120). For example, the optical imaging system 200 may apply a pressure or a vacuum to the un-labelled tissue in the cartridge 100. As a particular example, the optical imaging system 200 may control the pressure assembly 600 to apply pressure or a vacuum to the un-labelled tissue 160. In this case, the optical imaging system 200 may determine pressure or vacuum information, and control and apply a particular pressure or a vacuum to each pin 610 and pad 620 group of the pressure assembly 600 such that a consistent, or varied, pressure or vacuum may be applied to the surface of the un-labelled tissue 160.

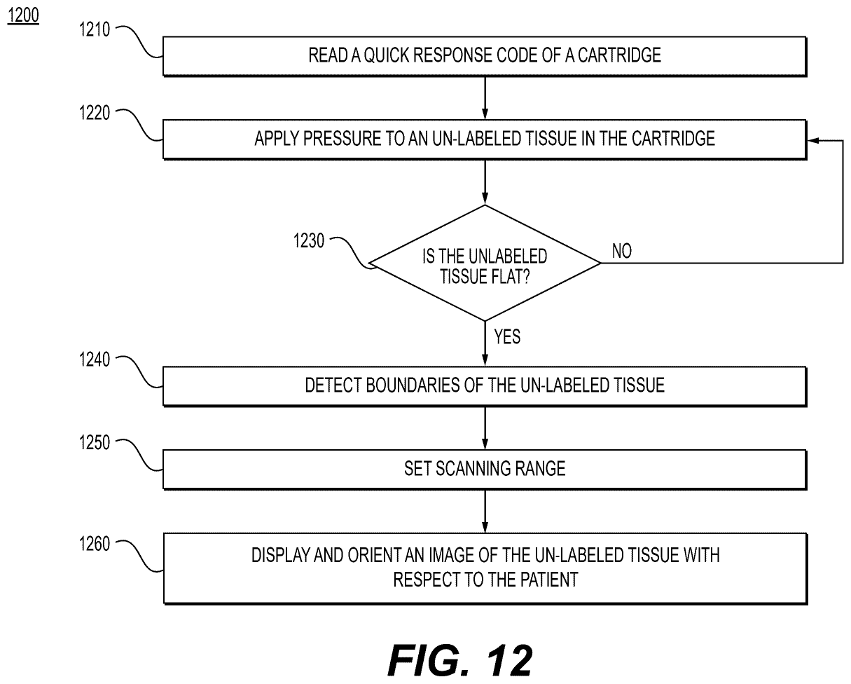

FIG. 12 is a flowchart of a process for imaging a tissue. As shown in FIG. 12, a process 1200 may include reading a quick response code of a cartridge 100 (operation 1210). For example, the optical imaging system 200 may read a QR code provided on the cartridge 100 and perform operations 1220 through 1260 based on reading the QR code.

As further shown in FIG. 12, the process 1200 may include applying a pressure to the un-labelled tissue 160 in the cartridge 100 (operation 1220). For example, the optical imaging system 200 may apply pressure to the un-labelled tissue 160. As a particular example, the optical imaging system 200 may determine pressure information, and control each pin 610 and pad 620 group of the pressure assembly 600 to apply a consistent, or varied, pressure to the surface of the un-labelled tissue 160. With the visible camera (210), the system can see if there is air between the optical substrate and the tissue. Alternatively, the scattering image from the imaging head (220) will give a very clear indication (usually very high signal) that the tissue is not flat against the optical substrate. In the example, when the scattering image in 210 picks up an air pocket after a full scan, more pressure (or vacuum) can be applied to the areas of the air pocket to get rid of the air and make the tissue against sample and the system can re-scan that region. In this case the newly scanned region can be stitched back to the full scan to create a full no-air pocket image.

As further shown in FIG. 12, the process 1200 may include determining whether the un-labelled tissue is flat (operation 1230). For example, the optical imaging system 200 may determine whether the un-labelled tissue 160 is flat against the optical substrate 120. Again, herein, “flat” may refer to a surface of the un-labelled tissue 160 being in contact with a surface of the optical substrate 120 (e.g., the top surface 120-1) such that no gap exists between the surface of the un-labelled tissue 160 and the surface of the optical substrate 120. The optical imaging system 200 may determine that the un-labelled tissue 160 is flat based on images captured by the optical imaging system 200, based on detecting that no gap exists between the surface of the un-labelled tissue 160 and the surface of the optical substrate 120, based on determining that no bubbles exist between the surface of the un-labelled tissue 160 and the surface of the optical substrate 120, based on applying a particular pressure to the un-labelled tissue 160, or the like.

As further shown in FIG. 12, if the un-labelled tissue is not flat (operation 1230—NO), then the process 1200 may include returning to operation 1220. For example, the optical imaging system 200 may iteratively adjust the pressure applied to the un-labelled tissue 160 until the un-labelled tissue 160 is flat against the optical substrate 120.

As further shown in FIG. 12, the process 1200 may include detecting boundaries of the un-labelled tissue (operation 1240). For example, the optical imaging system 200 may detect boundaries of the un-labelled tissue 160 using the camera head 210 and/or the imaging head 220. In the case where the imaging head 220 is used to detect boundaries, a low res scattering image can be used which will be fast.

As further shown in FIG. 12, the process 1200 may include setting a scanning range (operation 1250). For example, the optical imaging system 200 may set a scanning range of the optical imaging system 200 based on the detected boundaries of the un-labelled tissue 160.

As further shown in FIG. 12, the process 1200 may include displaying and orienting an image of the un-labelled tissue with respect to the patient (operation 1260). For example, the optical imaging system 200 may display, via the display 320, the tissue image 330, the tissue image axes 340, the patient image 350, the tissue image 360, and/or the direction indicator 370 in a similar manner as described above in connection with FIG. 3B.

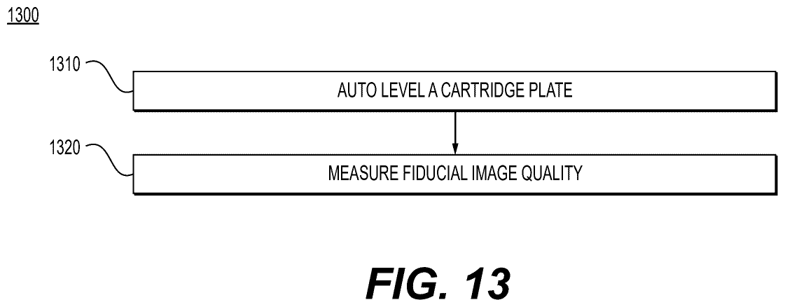

FIG. 13 is a flowchart of a process for adjusting an imaging head of an optical imaging system. As shown in FIG. 13, a process 1300 may include auto leveling a cartridge plate (operation 1310). For example, the optical imaging system 200 may measure the fiducials 700 in all four quadrants, and auto level the cartridge plate 230 relative to the beam to ensure the focal plane is consistent across the entire field of view of the un-labelled tissue 160 during a scanning acquisition.

As further shown in FIG. 13, the process 1300 may include measuring fiducial image quality (operation 1320). For example, by using scattering images from both excitation and detection lasers, the fiducials 700 may be optically resolved, and, if done at the start of each new PARS acquisition, the fiducial image quality can act as a beam health metric to ensure that the excitation and detection beams stay co-focused over time to avoid system drift. If the scattering image of either excitation or detection falls out or focus, the optical imaging system 200 may trigger an alarm that indicates that the optical imaging system 200 needs realignment.

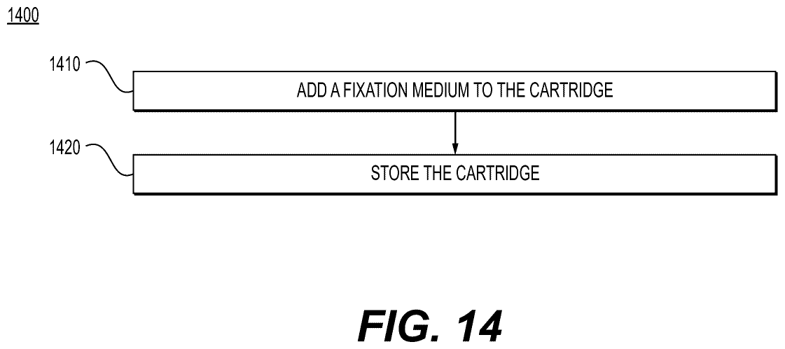

FIG. 14 is a flowchart of a process for storing a tissue in a cartridge. As shown in FIG. 14, a process 1400 may include adding a fixation medium to a cartridge (operation 1410), and storing the cartridge (operation 1420). For example, a fixation medium (e.g., formalin, paraffin, etc.) may be added to the cartridge 100, and the cartridge 100 may be stored for later usage, additional imaging, etc. In some implementations, the system can automatically detect bubbles, perform analysis of bubbles, and provide an instruction to the user how he or she should adapt the mechanical fixation of the sample to mount the tissue better.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the embodiments are not to be considered as limited by the foregoing description.

1. A system for optical imaging of an un-labelled tissue, the system comprising:
an optical imaging system configured to image the un-labelled tissue to generate a virtually-stained histological image of the un-labelled tissue or perform molecular detection or diagnosis; and
a cartridge for storing the un-labelled tissue to be imaged by the optical imaging system, the cartridge comprising:
a container to store the un-labelled tissue, and to interface with the optical imaging system;
an optical substrate, provided on a bottom surface of the container, through which the optical imaging system is configured to image the un-labelled tissue; and
a lid, provided on a top surface of the container, including a membrane to compress the un-labelled tissue against the optical substrate such that an entire margin of the un-labelled tissue is flat against the optical substrate.

2. The system of claim 1, wherein the cartridge includes orientation marks that permit maintenance of an orientation of the un-labelled tissue relative to a patient from which the un-labelled tissue is resected.

3. The system of claim 2, wherein the orientation marks are provided on the optical substrate.

4. The system of claim 1, wherein the cartridge includes a unique identifier.

5. The system of claim 4, wherein the unique identifier is provided on the container.

6. The system of claim 5, wherein the unique identifier is a quick response code.

7. The system of claim 4, wherein the unique identifier is an interlock for the optical imaging system.

8. The system of claim 4, wherein the unique identifier prevents the cartridge from being used with another un-labelled tissue.

9. The system of claim 4, wherein the unique identifier includes information identifying a pressure and/or a vacuum to apply to the cartridge to compress the un-labelled tissue against the optical substrate.

10. The system of claim 1, wherein the cartridge includes fiducials for measurement by the optical imaging system.

11. The system of claim 1, wherein the optical substrate includes a Fabry-Pérot etalon.

12. The system of claim 1, wherein the optical imaging system is a photon absorption remote sensing imaging system configured to generate radiative, non-radiative, and scattering effects in the un-labelled tissue.

13. The system of claim 1, further comprising:
a pressure assembly configured to apply pressure or vacuum to compress the tissue against the optical substrate.

14. (canceled)

15. (canceled)

16. (canceled)

17. (canceled)

18. A system for optical imaging of an un-labelled tissue, the system comprising:
a photon absorption remote sensing imaging system configured to image the un-labelled tissue to generate a virtually-stained histological image of the un-labelled tissue; and
a cartridge for storing the un-labelled tissue to be imaged by the photon absorption remote sensing imaging system, the cartridge comprising:
a container to store the un-labelled tissue, and to interface with the imaging system;
an optical substrate, provided on a bottom surface of the container, through which the imaging system is configured to image the un-labelled tissue; and
a lid, provided on a top surface of the container, including a membrane to compress the un-labelled tissue against the optical substrate such that an entire margin of the un-labelled tissue is flat against the optical substrate.

19. The system of claim 18, wherein the virtually-stained histological image emulates hematoxylin and eosin stains.

20. The system of claim 18, wherein the virtually-stained histological image is generated intraoperatively.

21. A cartridge for storing an un-labelled tissue to be imaged by an optical imaging system, the cartridge comprising:
a container to store the un-labelled tissue, and to interface with the optical imaging system configured to image the un-labelled tissue to generate a virtually-stained histological image of the un-labelled tissue;
an optical substrate, provided on a bottom surface of the container, through which the optical imaging system is configured to image the un-labelled tissue; and
a lid, provided on a top surface of the container, including a membrane to compress the un-labelled tissue against the optical substrate such that an entire margin of the un-labelled tissue is flat against the optical substrate.

22. (canceled)

23. (canceled)

24. (canceled)

25. (canceled)

26. (canceled)

27. (canceled)

28. (canceled)

29. (canceled)

30. The cartridge of claim 21, wherein the cartridge includes fiducials for measurement by the optical imaging system.

31. (canceled)

32. The cartridge of claim 21, wherein the optical imaging system is a photon absorption remote sensing imaging system configured to generate radiative, non-radiative, and scattering effects in the un-labelled tissue.

33. The cartridge of claim 21, wherein the virtually-stained histological image emulates hematoxylin and eosin stains.

34. (canceled)

35. (canceled)
